(19) United States
(12) Patent Application Publication
(10) Pub. No.: US 2024/0252784 A1
(43) Pub. Date: Aug. 1, 2024

(54) INTERMITTENT URINARY CATHETER HAVING A FOLDED, COMPACT CONFIGURATION

(71) Applicant: Hollister Incorporated, Libertyville, IL (US)

(72) Inventors: Luis Martins, Ballina (IE); William K. Arnold, Gurnee, IL (US); Damien Biggins, Ballina (IE); Jerome A. Henry, Castlebar (IE); Michael G. Murray, Ballina (IE); Vincent Naughton, Sligo (IE); Jeffrey R. Burger, Bexley, OH (US); Chris W. Cicenas, Etna, OH (US)

(21) Appl. No.: 18/560,502

(22) PCT Filed: May 10, 2022
(86) PCT No.: PCT/US2022/028492

§ 371 Date: Nov. 13, 2023

Related U.S. Application Data
(60) Provisional application No. 63/189,814, filed on May 18, 2021.

(51) Int. Cl. A61M 25/00; A61M 25/01

(52) U.S. Cl. CPC A61M 25/002; A61M 25/0017; A61M 25/0054; A61M 25/01; A61M 2025/0175; A61M 2209/06; A61M 2210/1089

(57) ABSTRACT

A urinary catheter assembly (10) having a folded, compact configuration and a straight ready to use configuration, having an outer tube (30) and an inner catheter tube (12), the inner catheter tube comprising a bendable region (24), the inner catheter tube and the outer tube being movable relative to one another so that a bending element of the bendable region of the inner catheter tube is moved into the lumen of the outer tube, thereby straightening the inner catheter tube so as to place the urinary catheter assembly into a straight ready to use configuration.

### FIELD OF THE DISCLOSURE

This disclosure generally relates to urinary catheter assemblies that have a compact configuration for distribution, storage, and convenient and discreet transport. More particularly, this disclosure relates to intermittent urinary catheter assemblies that have a compact, folded or bent configuration.

### BACKGROUND

Catheters are used to treat many different types of medical conditions and typically include an elongated catheter tube that is inserted into and through a passageway or lumen of the body. Urinary catheters and, in particular, intermittent urinary catheters are commonly used by individuals who suffer from certain abnormalities of the urinary system, such as urinary incontinence. With the advent of intermittent urinary catheters, individuals with problems associated with the urinary system can conveniently self-catheterize to drain the individual's bladder.

Individuals who suffer from urinary incontinence will self-catheterize several times a day. Thus, they are required to carry intermittent urinary catheters with them wherever they go. Accordingly, it is desirable for urinary catheters to have compact and portable configurations. It is also desirable for the catheters to be in configurations that allow the catheters to be discrete.

There remains a need for improved catheter products that are compact, portable, and/or discreet.

### SUMMARY

In one aspect a urinary catheter assembly includes an inner catheter tube partially positioned within a lumen of an outer tube. The inner catheter tube includes a bendable region that is at least partially outside of the outer tube and is bent so as to place the catheter assembly into a folded configuration. The inner catheter tube and the outer tube are movable relative to one another so that the bendable region of the inner catheter tube is moved into the lumen of the outer tube, thereby straightening the inner catheter tube so as to place the catheter assembly into a straight ready to use configuration. The outer tube and inner catheter tube being configured to be inserted into the urethra.

### BRIEF DESCRIPTION OF THE DRAWINGS

FIG. 1 is a side elevational view of one alternative of a urinary catheter product in accordance with the present disclosure;

FIG. 2 is a side elevational view of the catheter assembly of FIG. 1, shown in the straight configuration;

FIG. 3 is a side elevational view of another alternative of a urinary catheter assembly in accordance with the present disclosure;

FIG. 4 is a side elevational view of the catheter assembly of FIG. 3, shown in the straight configuration;

FIG. 5 is a partial side view of an alternative bendable region of the catheters of the present disclosure;

FIG. 6 is a partial side view of another alternative bendable region of the catheters of the present disclosure;

FIG. 7 is a partial side view of another alternative bendable region of the catheters of the present disclosure;

FIG. 8 is a partial side view of another alternative bendable region of the catheters of the present disclosure;

FIG. 9 is a partial side view of another alternative bendable region of the catheters of the present disclosure;

FIG. 10 is a partial side view of another alternative bendable region of the catheters of the present disclosure;

FIG. 11 is a side view of an alternative urinary catheter assembly of the present disclosure;

FIG. 12 is a partial side view of another alternative bendable region of the catheters of the present disclosure;

FIG. 13 is a partial side view of another alternative bendable region of the catheters of the present disclosure;

FIG. 14 is a perspective view of an alternative urinary catheter assembly of the present disclosure;

FIG. 15 is a perspective view of the catheter assembly of FIG. 14;

FIG. 16 is a partial perspective view of the distal end portion of the catheter assembly of FIG. 14;

FIG. 17 is a partial perspective view of an activation member of the catheter assembly of FIG. 14;

FIG. 18 is a perspective view of one alternative of a urinary catheter assembly;

FIG. 19 is a perspective view of the urinary catheter of FIG. 18;

FIG. 20A is a partial cross-sectional view of an activation member of the urinary catheter of FIG. 18;

FIG. 20B is a perspective view of an alternative activation member;

FIG. 20C is a cross-sectional view of the activation member of FIG. 20B;

FIG. 20D is an perspective exploded view of the activation member of FIG. 20B;

FIG. 21 is a side view of one alternative of a urinary catheter assembly;

FIG. 22 is a side view of the urinary catheter assembly of FIG. 21, shown in the straight configuration;

FIG. 23 is a side view of one alternative of a urinary catheter assembly;

FIG. 24 is a side view of the urinary catheter assembly of FIG. 23, shown in the straight configuration;

FIG. 25 is a side view of one alternative of a urinary catheter assembly;

FIG. 26 is a side view of the urinary catheter assembly of FIG. 25, shown in the straight configuration;

FIG. 27 is a side view of one alternative of a urinary catheter assembly;

FIG. 28 is a side view of the urinary catheter assembly of FIG. 27, shown in the straight configuration;

FIG. 29 is a side view of the urinary catheter assembly of FIG. 27, shown in the straight configuration with the sleeve collapsed;

FIG. 30 is a side view of one alternative of a urinary catheter assembly;

FIG. 31 is a side view of the urinary catheter assembly of FIG. 30, shown in the straight configuration;

FIG. 32 is a side view of one alternative of a urinary catheter assembly;

FIG. 33 is a side view of the urinary catheter assembly of FIG. 32, shown in the straight configuration and with the sleeve collapsed;

FIG. 34 is a side view of one alternative of a urinary catheter assembly;

FIG. 35 is a side view of the urinary catheter assembly of FIG. 34, shown in the straight configuration;

FIG. 36 is a side view of the urinary catheter assembly of FIG. 35, shown in the straight configuration with the sleeve collapsed;

FIG. 37 is a side view of another alternative urinary catheter assembly;

FIG. 38 is a side view of another alternative urinary catheter assembly;

and

FIG. 39 is a partial side view of the urinary catheter assembly of FIG. 37.

### DETAILED DESCRIPTION OF THE EMBODIMENTS

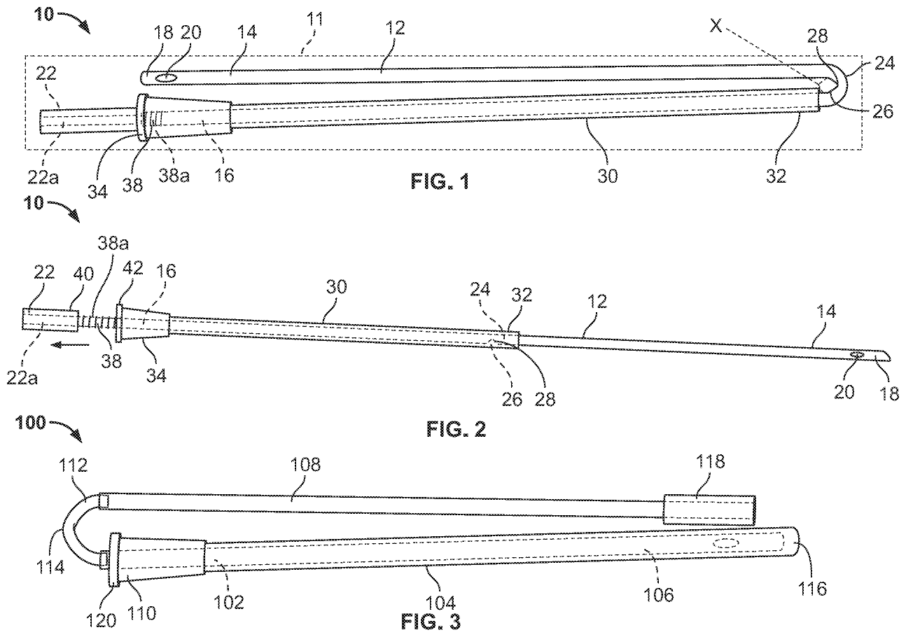

FIGS. 1 and 2 illustrate one embodiment of urinary catheter product that includes a catheter assembly 10 having an inner catheter tube 12 partially positioned within a lumen of an outer tube or sleeve 30, wherein the portion of the inner catheter tube 12 within the outer tube 30 is coaxial therewith. FIG. 1 shows the catheter assembly 10 in a bend or folded, compact configure for distribution, storage, and transport. Optionally, the catheter assembly 10 may be provided to the user in a package 11. The inner and outer catheter tubes 12, 30 may include a lubricious hydrophilic coating on the outer surface thereof and the package 11 may include a hydration medium, such as liquid or vapor water, for hydrating the coating. FIG. 2 shows the catheter assembly 10 in a straight configuration that is ready for use. It will be understood that due to the nature and flexible characteristics of the material of the catheter tube, when the catheter assembly is in the straight configuration, it may be straight or substantially straight or have a slight curvature or bend.

Catheter assembly 10 includes an elongated inner catheter tube 12 having a proximal insertion end portion 14 and a distal end portion 16. Proximal insertion end portion 14 includes a tip region 18 that is suitable for insertion into the urethra of a user. The tip region 18, optionally, may include draining holes or eyes 20 for receiving urine therethrough and into an internal conduit or drainage lumen of inner catheter tube 12. A drainage member 22 is associated with the distal end portion 16 of the inner catheter tube 12. The drainage member 22 has a drainage lumen 22a in communication with the drainage lumen of the inner catheter tube 12. The drainage member 22 may be a funnel or a connector that is configured to connect the inner catheter tube 12 to a collection receptacle, such as a urine collection bag.

The inner catheter tube 12 also includes a bending or bendable region 24 for bending or folding the inner catheter tube 12 into a compact configuration, thereby placing the catheter assembly 10 in a folded, compact configuration. The bendable region 24 includes a bending element 26. The bendable region 24 and the bending element 26 may reduce the amount of stress placed on the inner catheter tube 12 and outer tube 30. When in compact configuration, the bendable region 24 is at least partially outside of the outer tube 30 and/or at least partially within the outer tube 30. Optionally, the bendable region 24 may have a portion that is located within the outer tube 30 and a portion that extends out of the proximal end of the outer tube 30, wherein the end of the outer tube may act as a fulcrum for the bendable region 24/bending element 26.

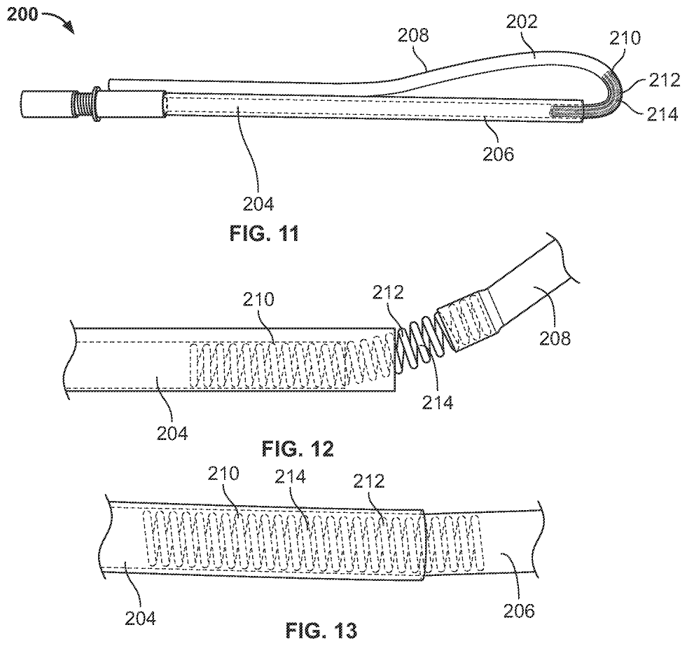

In the illustrated embodiment, the bending element 26 is a cut-out or notch 28 in the inner catheter tube 12. Such notches 28 may be formed by blades, lasers, dissolving material or melting. As shown in the FIGS. 3-4 and FIGS. 5-10, the bending element 26 may be any suitable element that assists in bending the inner catheter tube 12. Turning first to FIGS. 1, 2 and 5, the bendable region 24 may have a bending element 26 that includes one or more notches 28. The notch(es) 28 may have any suitable shape, such as the illustrated V-shaped side profile. Referring to FIG. 6, the bending element 26a may be a notch 28a having a generally rectangular side profile. Turning to FIG. 7, the bending element 26b may be one or more longitudinal slits 28b. As shown in FIG. 8, the bending element 26c may be a notch 28c having a generally semi-circular side profile. As illustrated in FIG. 9, the bending element 26d include one or more arched slits 28d partially about the circumference of the inner catheter tube 12. Turning FIGS. 3, 4 and 10 the bendable region 112 and bending element 114 may be a separate component or length of tubing 113 that is located between the proximal and distal end portions of the inner catheter tube 102. In other alternatives, the bendable region, and the bending element by be a thinner material than other portions of the inner catheter tube 12, chemically treated material or material of a flexibility different from other material of the inner catheter tube 12. For example, the bendable region may include heat stretch/shrink sleeves. The bendable region may be chemically or physically transformed. For example, the bendable regions may be heat stretched or physically abraded to reduce wall thickness. Such abrasion may be around the entire circumference of the bendable region or in discrete locations. As shown in FIGS. 11-13 and described in greater detail below, the bending element of the bendable region may include a bendable support structure, such as a spring or elastic struts, strips, or rods.

As mentioned above, the catheter assembly 10 also includes an outer tube 30 having a proximal end 32, a distal end 34 and a lumen extending therebetween. The distal end portion 16 of the inner catheter tube 12 is coaxially positioned within the lumen of the outer tube 30 and the proximal end portion 14 of the inner tube extends out of the proximal end 32 of the outer tube 30. A segment of the distal end portion 16 and/or drainage member 22 extends out of the distal end 34 of the outer tube 30.

Referring to FIG. 1, in the folded, compact configuration, the inner catheter tube 12 of the catheter assembly 10 is positioned within the outer tube 30 such that the bendable region 24 and bending element 26 are at least partially outside of the proximal end 32 of the outer tube 30. Optionally, a distal portion of the bendable region 24 and bending element 26 may be located within the outer tube 30, or the bendable region 24 and bending element 26 may be completely outside of the outer tube 30. Additionally, the bendable region 24 is adjacent to the proximal end 32 of the outer tube 30. In this position, the bendable region 24 can be bent such that the inner catheter tube 12 is in a folded configuration and the catheter assembly 10 is in a folded, compact configuration. In the illustrated embodiment, the proximal end portion 14 of the inner catheter tube 12 is folded back over towards the distal end portion 16. In other words, the inner catheter tube 12 is folded back over itself. In one embodiment, the proximal end portion 14 of the inner catheter tube 12 and the distal end portion 16 extend relative to each other from the bendable region 24. In one embodiment, the proximal end portion 14 and the distal end portion 16 extend relative to each other from the bendable region 24 at an angle X, that is greater than 90 degrees, and preferably greater than or equal 160 and less than 180 degrees.

Optionally, the catheter assembly 10 may be held or maintained in the compact configured by a package 11. For example, the size and shape of the package 11 may be such that the contact between the package walls and the catheter assembly 10 hold the catheter assembly 10 in the folded configuration. In another alternative, the package also could be foldable. In such an alternative, the package may extend and surround the longitudinal axial length of the catheter in the straightened configuration. Both the package and the catheter may then be in a folded configuration for distribution and storage prior to use. The package may be folded, (in half for example) such that the opposed longitudinal ends of the package are adjacent to each other. The opposed longitudinal ends may be attached or held together by a releasable adhesive or a tie, such as a rubber band.

Referring to FIG. 2, the catheter assembly 10 is shown in the straight configuration, in which the catheter assembly 10 is ready to use for catheterization. The inner catheter tube 12 and the outer tube 30 are moved relative to each other such that the bendable region 24 and the bending element 26 are moved to be substantially located within the outer tube 30. This moves the inner catheter tube 12 into a substantially straight configuration with the outer tube 30. The inner catheter tube 12 and outer tube 30 may be moved by the user pulling the inner catheter tube 12 distally relative to the outer tube 30. Alternatively, the catheter assembly 10 may include a biasing member 38 that biases the catheter assembly toward the straight configuration. In the illustrated embodiment, the biasing member 38 positioned between the inner catheter tube 12 and the outer tube 30. For example, the biasing member 38 may be a helical spring 38a positioned around a distal section of inner catheter tube 12 and between a proximal surface 40 of the drainage member 22 and a distal surface 42 of the outer tube 30. The distal surface 42 of the outer tube 30 may be a shoulder or other radially extending surface. Referring to FIG. 1, in the folded, compact configuration the spring 38a is compressed between the proximal surface 40 of the drainage member 22 and the distal surface 42 of the outer tube 30. In FIG. 2, the spring 38a forces the inner catheter tube 12 to move distally relative to the outer tube 30, such that the bendable region 24 and the bending element 26 move into the lumen of the outer tube 30, thereby straightening inner catheter tube 12 and putting the catheter assembly 10 into the straight configuration. The biasing member, optionally, may also aid in maintaining the catheter assembly in the straight configuration.

In use for catheterization of the bladder, the bendable region 24 of the inner catheter tube 12 is within the outer tube 30, and proximal end portion 14 of the catheter tube 102, the bendable region 24 and at least the proximal end 32 of the outer tube 30 may be inserted into the urethra. Thus, the bendable region 24 and proximal end 32 of the outer tube 30 are portions of the catheter assembly 10 that are insertable into the urethra. Optionally, a transition suitable for atraumatic insertion into the urethra is configured between to the proximal end 32 of the outer tube 30 and the surface of the inner catheter tube 12. For example, the transition may be a smooth transition. In another alternative, the proximal end 32 of the outer tube 30 may remain outside and/or be configured to remain outside of the urethra. That is, proximal end 32 is not inserted into the urethra.

After catheterization, optionally, the user may return the catheter assembly to the compact configuration. If desired, the catheter may be placed back into the package and disposed of.

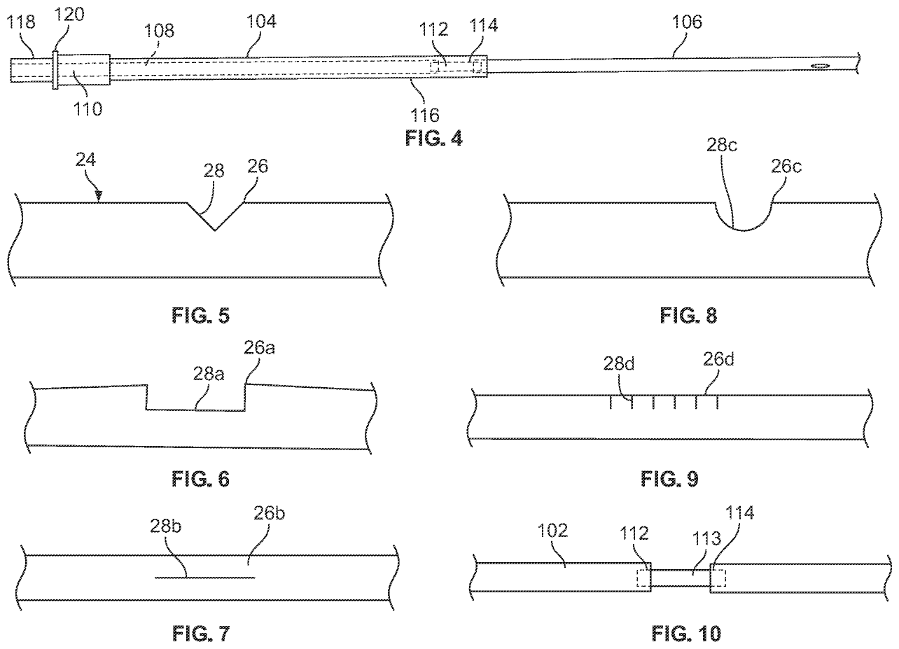

FIGS. 3 and 4 illustrate another alternative of the catheter assembly 100 of the present disclosure. FIG. 3 shows the catheter assembly 100 in a bent or folded compact configuration for distribution, storage, and transport. FIG. 4 shows the catheter assembly 100 in a straight configuration and ready for use.

The catheter assembly 100 includes an inner catheter tube 102 coaxially partially positioned within a lumen of an outer tube 104. In the compact configuration, the inner catheter tube 102 includes a proximal end portion 106 within the outer tube 104 and a distal end 108 extending out of the distal end 110 of the outer tube 104. The inner catheter tube 102 has a bendable region 112 that includes a bending element 114. In the illustrated embodiment, the bending element 114 is a segment of flexible tubing attached to the proximal and distal end portions 106 and 108 of the catheter tube 102. However, the bending element 114 may be any bending element disclosed herein. The bendable region 112 is at least partially outside of the outer tube 104 and/or at least partially within outer tube 104. The bendable region 112 is adjacent the distal end 110 of the outer tube 104. Optionally, the bendable region 112 may have a portion that is located within the outer tube 104 and a portion that extends out of the distal end 110 of the outer tube 104, wherein the end of the outer tube 104 may act as a fulcrum for the bendable region 112/bending element 114. Alternatively, the bendable region 112/bending element 114 may be completely outside of outer tube 104.

To move the catheter assembly 100 from the compact configuration to the straight ready to use configuration, the inner catheter tube 102 is slid proximally relative to the outer tube 104 such that the bendable region 112 and bending element 114 are moved proximally and at least partially into the outer catheter tube 104. The proximal end portion 106 of the inner catheter tube 102 is moved out of the proximal end 116 of the outer tube 104, as illustrated in FIG. 4. The inner catheter tube 102 may include a drainage member 118 associated with its distal end 108. Optionally, the drainage member 118 mates with a port 120 associated with the distal end 110 of the outer tube 104. This mating engagement may assist in maintaining the catheter assembly 100 in the straight configuration. As with the previous embodiment, during catheterization the bendable region 112 and proximal end 116 of the outer tube 104 may be inserted into the urethra. In another alternative, the proximal end of the outer tube may remain outside and/or be configured to remain outside of the urethra. After catheterization, the catheter assembly may be moved back into the compact, folded configuration.

FIGS. 11-13 illustrate further alternatives of the urinary catheter assembly 200. The urinary catheter assembly 200 may have the compact configuration shown in FIG. 1 wherein the distal end portion 204 of the inner catheter tube 202 is located within the outer tube 206. Alternatively, the urinary catheter assembly 200 may have the compact configuration shown in FIG. 2 wherein the proximal end portion 208 of inner catheter tube 202 is located within the outer tube 206.

As shown in these figures, the bending element 210 of the bendable region 212 includes a helical spring 214. In other alternatives, the bending element may be elastic struts, rods, or strips. Referring to FIG. 12, in one alternative the bending element 210 may be between and connect the proximal and distal end portions 208 and 204 of the inner catheter tube. Also in this alternative, the bending element 210 may include at least a portion that is exposed and not within the inner catheter tube 202. For example, one end of the bending element 210 may be inserted into the proximal end portion 208 of the inner catheter tube 202 and the other end of the bending element 210 may be inserted into the distal end portion 204 of the inner catheter tube 202. Alternatively, the proximal and distal end portions 208 and 204 of the inner catheter tube 202 may be inserted, respectively, into the ends of the bending element 210. Another alternative is illustrated in FIG. 13. In this alternative, the bending element 210 is located within the inner catheter tube 202 wherein the bending element 210 may provide support to the inner catheter tube 202.

Referring back to FIG. 11, in either alternative, when the catheter assembly 200 is in the compact configuration a portion of the bending element 210 is located within the outer tube 206 and another portion is located outside of outer tube 206. As with the previous embodiments, to move the catheter assembly 200 into the straight configuration, the inner catheter tube 202 and outer tube 206 are moved relative to one another to move the bending element 210 fully into the outer tube 206.

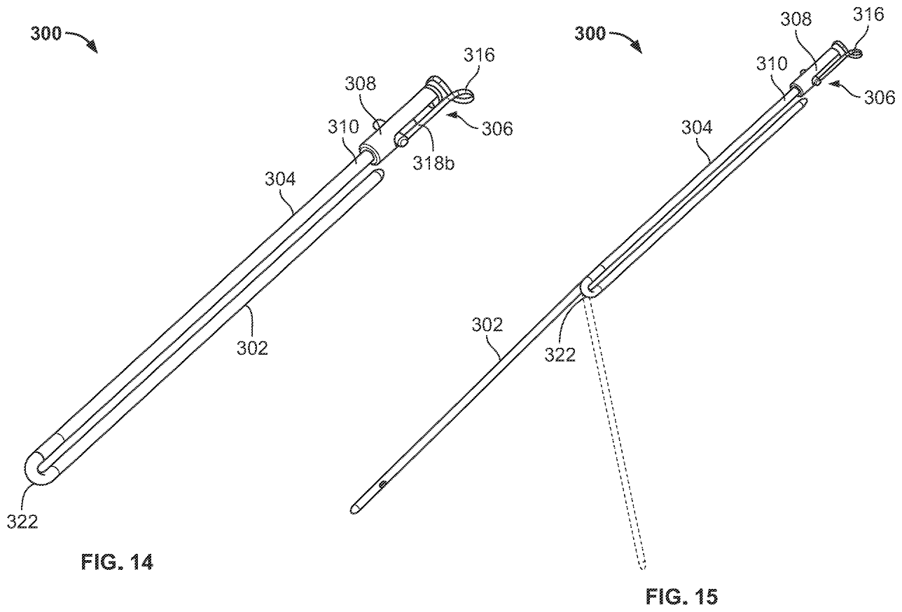

FIGS. 14-17 illustrate one alternative catheter assembly 300. Similar to the previous embodiments, the catheter assembly 300 includes an inner catheter tube 302 and an outer tube 304. The catheter assembly 300 also includes an activation mechanism 306 that when activated by the user moves the catheter assembly 300 from the compact configuration (FIG. 14) to the straight configuration (FIG. 15).

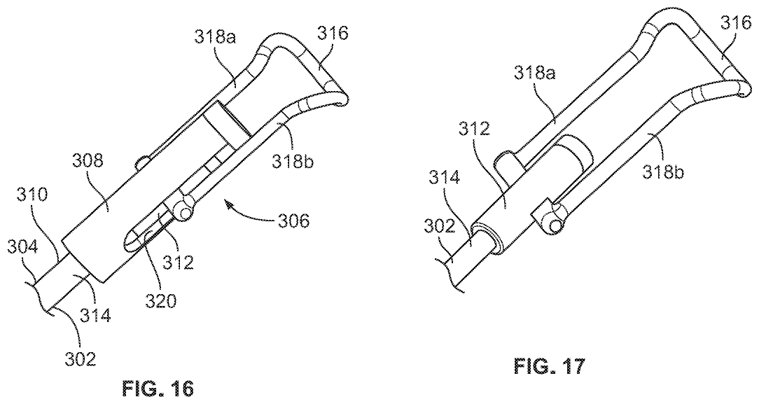

Referring to FIGS. 14 and 15, the activation mechanism 306 includes a housing 308 that is attached to the distal end 310 of outer tube 304. Turning to FIGS. 16 and 17, the activation mechanism 306 also includes a slider 312 positioned within the housing 308. Referring to FIG. 17, the slider 312 is attached to the distal end portion 314 of the inner catheter tube 302. A pull ring 316 is attached to the slider 312. In the illustrated embodiment, the pull ring 316 is generally U-shaped, wherein each leg 318a, 318b of the U is attached to the slider 312. As shown in FIG. 16, the legs 318a, 318b extend through elongated slots 320 in the housing 308 and are connected to the slider 312.

To move the catheter assembly 300 from the compact configuration to the straight configuration, the user activates the mechanism by pulling the pull ring 316 distally. This in turn moves the slider 312 distally and moves the inner catheter tube 302 distally relative to the outer tube 304. When the inner catheter tube 302 is moved distally, the bendable region 322 of the inner catheter tube 302 is moved into the outer tube 304, thereby straightening the inner catheter tube 302 and moving the catheter assembly 300 into the straight configuration.

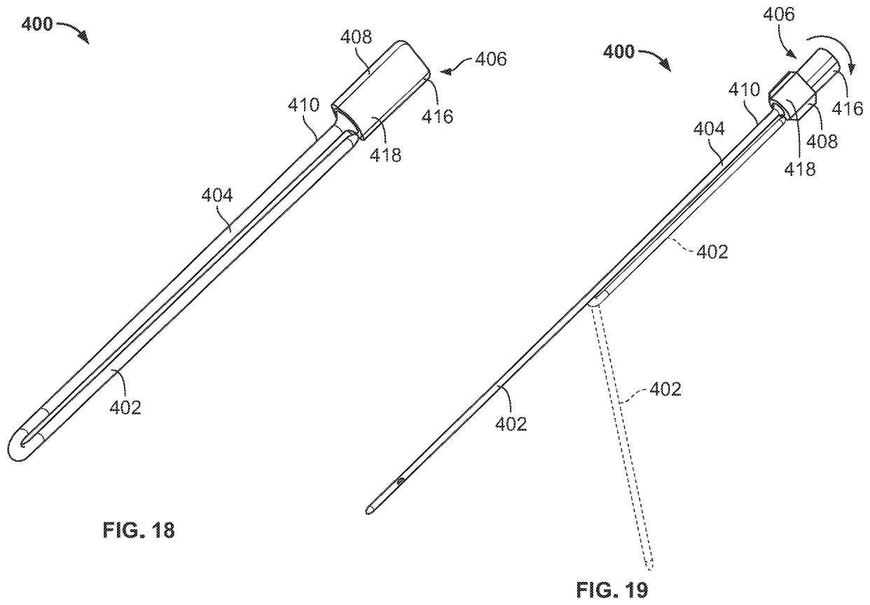

FIGS. 18-20A illustrate another alternative catheter assembly 400. Similar to the previous embodiments, the catheter assembly 400 includes an inner catheter tube 402 and an outer tube 404. The catheter assembly 400 also includes an activation mechanism 406 that when activated by the user moves the catheter assembly from the compact configuration (FIG. 18) to the straight configuration (FIG. 19).

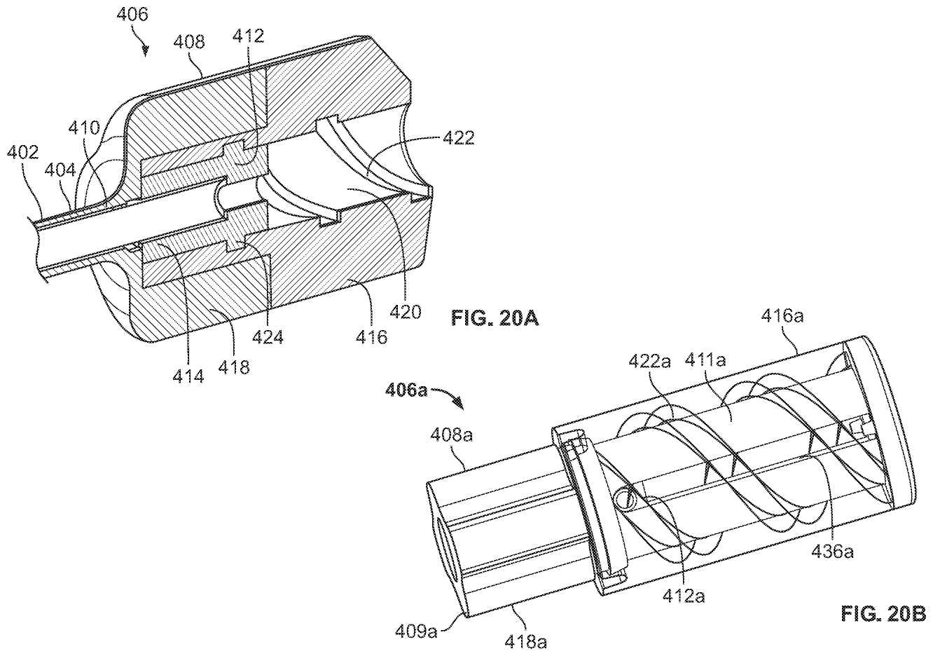

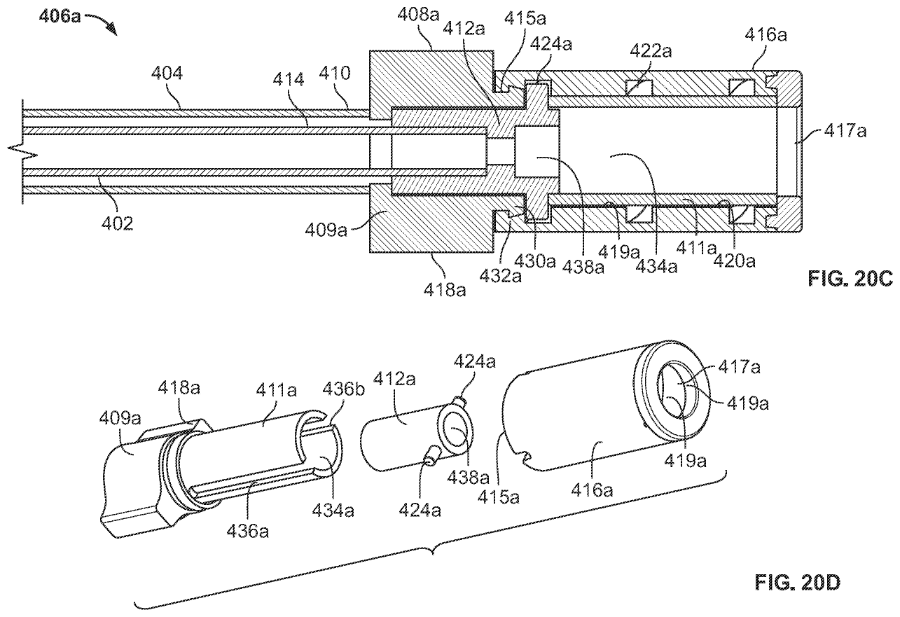

The activation mechanism 406 includes a housing 408 that is attached to the distal end 410 of outer tube 404. Turning to FIG. 20A, the activation mechanism 406 also includes a cam 412 positioned within the housing 408. The cam 412 is attached to the distal end portion 414 of the inner catheter tube 402. The housing 408 including a rotatable distal end portion 416 that rotates relative to the proximal portion 418 of the housing. The rotatable distal end portion 416 includes an inner surface 420 that has a spiral groove 422. The cam 412 includes a shoulder or post 424 that mates with the spiral groove 422 of the housing 408, such that when rotatable distal end portion 416 is rotated, the cam 412 moves axially within the housing 408.

To move the catheter assembly 400 from the compact configuration to the straight configuration, the user activates the activation mechanism 406 by rotating the rotatable distal end portion 416 of the housing 408 relative to the proximal end portion 418 of the housing 408. As the distal end portion 416 is rotated, the shoulder 424 of the cam 412 moves along the groove 422, thereby moving the cam 412 axially in a distal direction within the housing 408. This in turn moves the inner catheter tube 402 distally relative to the outer tube 404. When the inner catheter tube 402 is moved distally, the bendable region 425 of the inner catheter tube 402 is moved into the outer tube 404, thereby straightening the inner catheter tube 402 and moving the catheter assembly 400 into the straight configuration.

FIGS. 20B-20C illustrate an alternative activation member 406a that may be used with catheter assembly 400. The activation mechanism 406a includes a housing 408a that is attached to the distal end 410 of outer tube 404 (FIG. 20C). The housing 408a including a handle 418a having a proximal gripping portion 409a and distal section 411a. A cover 416a is positioned over a distal section 411a of the handle 418a. In the illustrated activation member 406a, the cover 416a includes a proximal opening 415a, a distal opening 417a, and a lumen 419a. To assemble the activation member 406a, the distal section 411a of the handle 418a is inserted through the distal opening 415a of the cover 416a and into the lumen 419a. The handle 418a includes a catch 430a the engages a latch 432a of the cover 416a to attach the handle 418a and cover 416a. The cover 416a and the handle 418a are rotatable relative to one another.

The activation mechanism 406a also includes a cam 412a positioned within the housing 408a. The cam 412a is attached to the distal end portion 414 of the inner catheter tube 402. The handle 418a includes a lumen 434a in which the cam 412a is located. First and second channels 436a, 436b are located through the wall of distal section 411a of the handle 418a and extend longitudinally at least partially along a distal section 411a. The cam 412a includes at least one post 424a that extends through the channels 436a, 436b and engage with spiral grooves 422a located on the inner surface 420a of cover 416a. When the cam 412a includes two posts 424a, the grooves 422a may include two grooves in a double helix configuration. When the handle 418a and cover 416a are rotated relative to one another, the cam 412a moves within to grooves 422a to move the cam 412a axially within the housing 408a, thereby moving the catheter tube.

Additionally, the cam 412a includes a lumen 438a therein which is in communication with the lumen of the catheter tube 402 and the lumen 434a of the handle and/or the lumen of the cover 416a. During catheterization, draining urine will flow from the catheter tube, through the lumen 438a of the cam 412a, through the lumen(s) of the handle and/or the cover, and out of the distal opening of the cover.

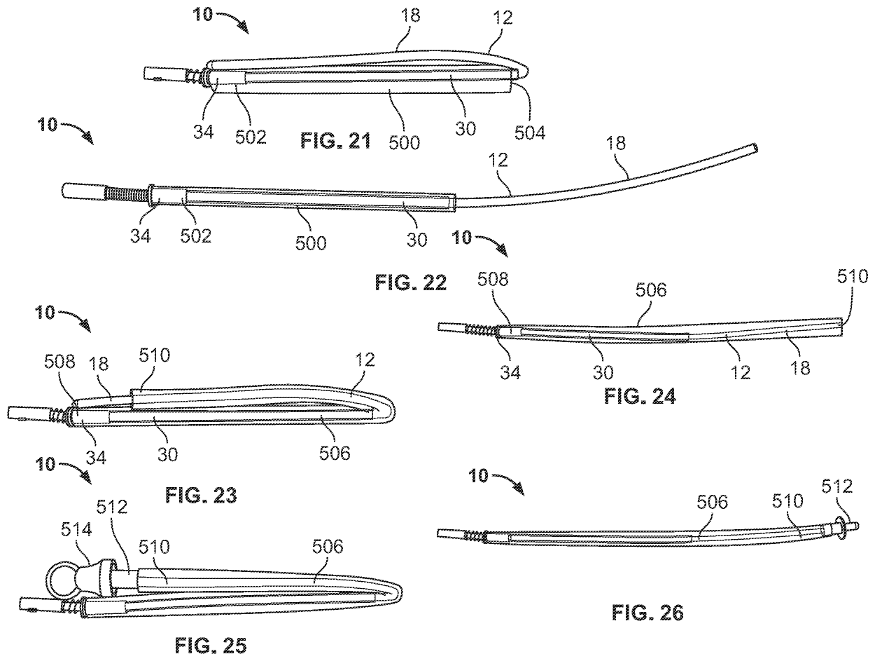

FIGS. 21-36 illustrate various sleeved catheter assemblies. Collapsible and/or flexible sleeves may be employed with any of the catheter assemblies disclosed herein. The catheter assemblies may include at least one sleeve extending over at least a portion of the inner catheter tube and/or at least a portion of the outer tube. The sleeve may be made from a thin plastic film or sheeting. The sleeves may provide a sterile barrier between the catheter assembly and the user. For example, the user may grasp and manipulate the catheter assembly through the sleeve during removal form the package, straightening of the catheter assembly and catheterization.

FIGS. 21 and 22 illustrate catheter assembly 10 (FIG. 1) that includes a flexible sleeve 500 extending over at least a portion of the outer tube 30. The distal end 502 of the sleeve 500 is attached to the distal end 34 of the outer tube 30 and the proximal end 504 is a free end. In the illustrated embodiment, the sleeve only extends over the outer tube 30 and does not extend over the proximal end portion 14 of the inner catheter tube 12.

In FIGS. 23 and 24, catheter assembly 10 (FIG. 1) includes a flexible and collapsible sleeve 506 having a length such that the sleeve 506 extends over the outer tube 30 and at least partially over the proximal end 14 of the inner catheter tube 12. The distal end 508 of the sleeve 506 may be attached to the distal end 34 of the outer tube 30. Additionally, the proximal end 510 of sleeve 506 may be a free end having an opening. When the proximal end 14 of the inner catheter tube 12 is inserted into the urethra the collapsible sleeve collapses against the meatus as the catheter is advanced into the urethra.

FIGS. 25 and 26 are similar to that of shown in FIGS. 23 and 24, except that an insertion aid 512 is attached to the proximal end 510 of the sleeve 506. Optionally, the catheter assembly 10 may include a cap 514 to cover the insertion aid 512. During catheterization, the insertion aid 512 is inserted into the urethral opening and the inner catheter tube 12, and possibly at least a portion of the outer tube 30, are advanced through the insertion aid 512 into the urethra. As the catheter is advanced the sleeve 506 collapses between the insertion aid 512 and the distal end of the outer tube 30.

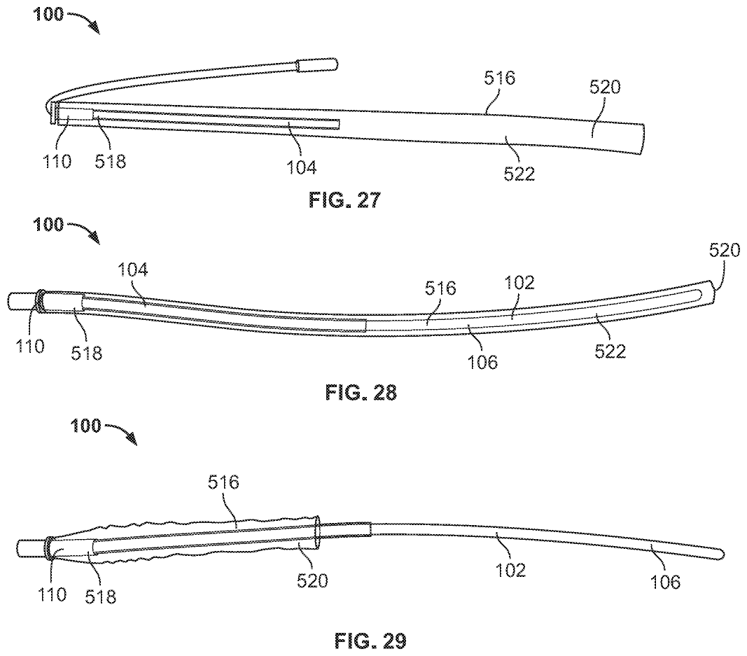

FIGS. 27-29 illustrate catheter assembly 100 (FIG. 3) that includes a flexible sleeve 516 extending over at least a portion of the outer tube 104. The distal end 518 of the sleeve 516 is attached to the distal end 110 of the outer tube 104 and the proximal end 520 of the sleeve 516 is a free end having an opening. In the illustrated embodiment, the sleeve 516 extends over the outer tube 104 and beyond its proximal end. Referring to FIG. 27, in the compact configuration, the sleeve 516 covers the outer tube 104 and a proximal portion 522 of the sleeve 516 beyond the outer tube does not contain the catheter tubes. Turning to FIG. 28, when the catheter assembly 100 is moved into the straight configuration, the proximal end 106 of the inner catheter tube 102 is moved out of the outer tube 104 and into the proximal portion 522 of the sleeve 516, wherein the sleeve 516 covers the proximal end 106 of the inner catheter tube 102. Referring to FIG. 29, as the catheter assembly 100 is inserted into the urethra, the sleeve 516 collapses distally.

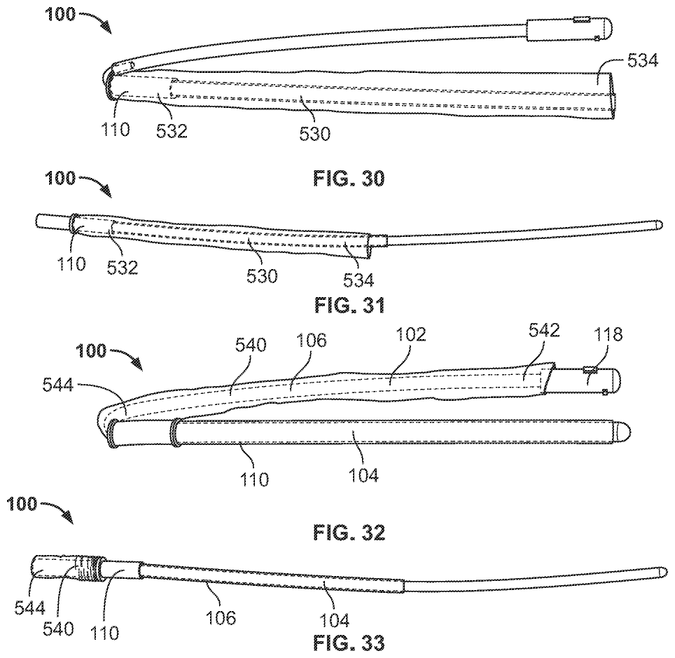

FIGS. 30 and 31 illustrate catheter assembly 100 (FIG. 3) that includes a flexible sleeve 530 extending over at least a portion of the outer tube 104. The distal end 532 of the sleeve is attached to the distal end 110 of the outer tube 104 and the proximal end 534 of the sleeve 530 is a free end having an opening. In the illustrated embodiment, the sleeve only extends over the outer tube 104.

FIGS. 32 and 33 illustrate catheter assembly 100 (FIG. 3) wherein a sleeve 540 extends over and covers the distal end 108 of the inner catheter tube 102. The distal end 542 of sleeve 540 may be attached to the drainage member 118, and/or optionally, to the distal end of the inner tube. The proximal end 544 of the sleeve 540 may be attached to distal end 110 of the outer tube 104. When the distal end portion 108 of the inner catheter tube 102 is moved into the outer tube 104, the sleeve 540 collapses between the drainage member 118 and distal end 110 of the outer tube 104.

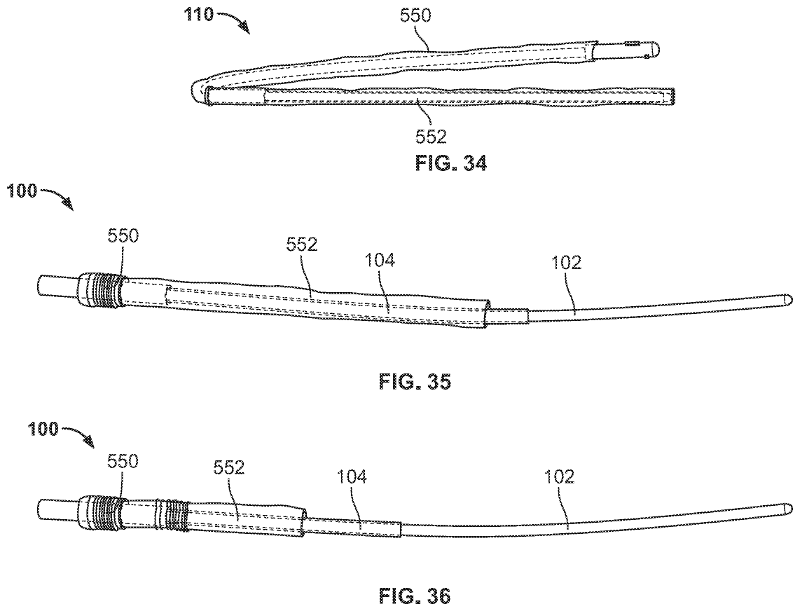

FIGS. 34-36 illustrate catheter assembly 100 (FIG. 3) wherein a first sleeve 550 extends over and covers the distal end 108 of the inner catheter tube 102 and a second sleeve 552 extends over at least a portion of the outer tube 104. In one embodiment the second sleeve 552 extends beyond the outer tube. The sleeve 550 and 552 may be attached in any similar fashion described above. Referring to FIG. 35, when the inner catheter tube 102 is moved proximally within outer tube 104 to place the catheter assembly 100 in the straight configuration, the sleeve 550 collapses. Referring to FIG. 36, when the catheter assembly 100 is inserted into the urethra, the sleeve 552 collapses. Also, optionally, a single sleeve may extend over the distal end of the inner catheter tube and over at least a portion of the outer tube.

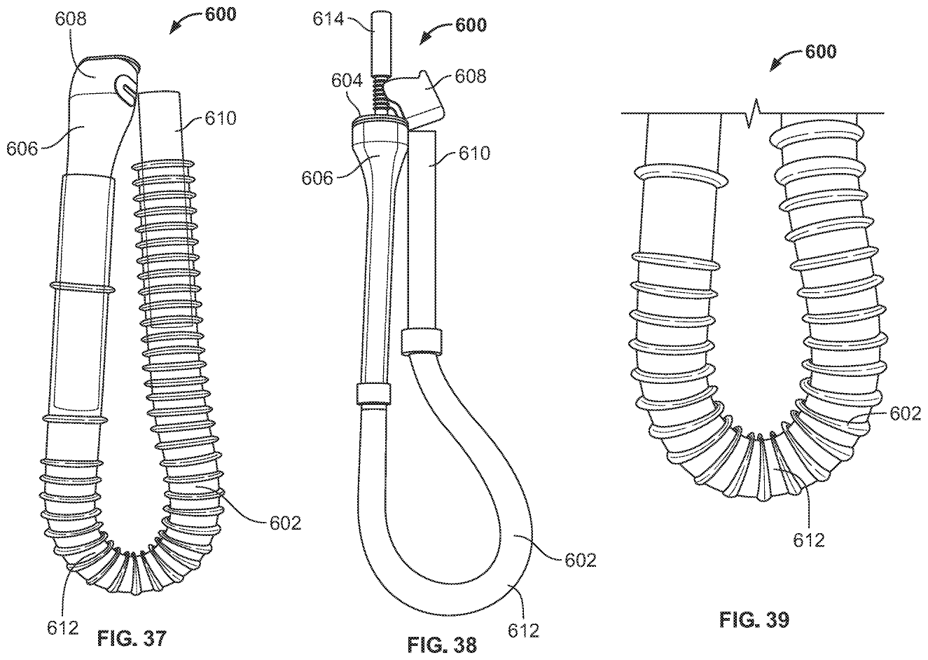

FIGS. 37-39 illustrate a packaging 600 for the catheter assemblies disclosed herein. The packaging 600 includes a housing 602, which may be a hose, that has a generally U-shaped configuration. The housing 602 includes an opening 604 (FIG. 38) at one end 606 thereof. The opening 604 may be closed by a lid or cap 608. The opening 604 may be a reclosable opening that is reclosable the cap 608. The other end thereof 610 is a closed end. In the illustrated embodiment, the cap 608 is hingedly connected to the housing.

The housing 602 includes a flexible middle section 612 that defines the bottom of the U-shaped housing 602. The flexible middled section 612 may be a corrugated as shown in FIGS. 37 and 39 or otherwise a flexible material as shown in FIG. 38. To remove the catheter 614 from the housing 602, the user removes the lids 608 and pulls the catheter 614 out of the housing 602.

It should be understood that various changes and modifications to the presently preferred embodiments described herein will be apparent to those skilled in the art. Such changes and modification can be made without departing from the spirit and scope of the invention disclosed herein.

What is claimed is:

1. A urinary catheter product, comprising:
a urinary catheter assembly comprising:
outer tube having a lumen;
an inner catheter tube partially positioned within the lumen of the outer tube, the inner catheter tube including a bendable region having a bending element, the bendable region being at least partially outside of the outer tube and bent so as to place the catheter assembly into a folded configuration;
the inner catheter tube and the outer tube being movable relative to one another so that a bending element of the bendable region of the inner catheter tube is moved into the lumen of the outer tube, thereby straightening the inner catheter tube so as to place the urinary catheter assembly into a straight ready to use configuration; and
wherein the outer tube and inner catheter tube are configured to be inserted into a urethra.

2. The urinary catheter product of claim 1, wherein the inner catheter tube has a proximal end portion and a distal end portion, and the bendable region is between the proximal end portion and the distal end portion.

3. The urinary catheter product of claim 2, wherein the bendable region is a separate component connected to the proximal end portion and the distal end portion of the inner catheter tube.

4. The urinary catheter product of claim 2, wherein when the catheter assembly is in the folded configuration, the distal end portion of the inner catheter tube is located within the outer tube.

5. The urinary catheter product of claim 1, wherein when the catheter assembly is in the folded configuration, the bendable region is adjacent a proximal end portion of the outer tube.

6. The urinary catheter product of claim 2, wherein when the catheter assembly is in the folded configuration, the proximal end portion of the inner catheter tube is located within the outer tube.

7. The urinary catheter product of claim 1, wherein when the catheter assembly is in the folded configuration, the bendable region is adjacent a distal end portion of the outer tube.

8. The urinary catheter product of claim 1, wherein the catheter assembly includes a biasing member that biases the catheter assembly toward the straight ready to use configuration.

9. The urinary catheter product of claim 8, wherein the biasing member comprises a spring.

10. The urinary catheter product of claim 9, wherein the spring is positioned between a distal end of the outer tube and the inner catheter tube.

11. The urinary catheter product of claim 1, wherein the bending element comprises a spring.

12. The urinary catheter product of claim 1, wherein the bending element comprises a notch.

13. The urinary catheter product of claim 1, wherein the bending element comprises a slit.

14. The urinary catheter product of claim 1, further including at least one sleeve extending over at least a portion of the inner catheter tube and/or at least a portion of the outer tube.

15. The urinary catheter product of claim 14, wherein the sleeve extends over the outer tube.

16. The urinary catheter product of claim 15, wherein the sleeve extends proximally beyond a proximal end of the outer tube.

17. The urinary catheter product of claim 15, wherein the sleeve extends over a portion of the inner catheter tube.

18. The urinary catheter product of claim 14, wherein the at least one sleeve comprises a first sleeve and a second sleeve, wherein the first sleeve extends over the outer tube and the second sleeve extends at least a portion of the inner catheter tube.

19. The urinary catheter product of claim 1, further including a packaging, wherein the catheter assembly in the folded configuration is located in the packaging.

20. The urinary catheter product of claim 19, wherein the packaging is configured to maintain the urinary catheter assembly in the folded configuration.

21. The urinary catheter product of claim 1, wherein the urinary catheter assembly further includes a activation mechanism for moving the urinary catheter assembly from the folded configuration to the straight ready to use configuration.

22.-23. (canceled)
